(19) United States
(12) Patent Application Publication
(10) Pub. No.: US 2009/0270946 A1
(43) Pub. Date: Oct. 29, 2009

(54) Therapeutic Light System

(76) Inventor: Paul Spivak, Chesterland, OH (US)

Correspondence Address: PATENT, COPYRIGHT & TRADEMARK LAW GROUP, 4199 Kinross Lakes Parkway, Suite 275, RICHFIELD, OH, 44286, US

(21) Appl. No.: 12/110,058

(22) Filed: Apr. 25, 2008

(51) Int. Cl. A61N 5/06; A61N 1/00

(52) U.S. Cl. 607/54; 607/88

(57) ABSTRACT

A non-thermal low dose light emitting diode (LED) array provides for the application of therapeutically specific radiation is provided. The array delivers radiation to the intended target surface at a controlled intensity. The intensity is controlled with a specific sequence of pulsing by pulse driving a matrix-arrayed LED. The modulation of the therapeutically specific radiation is at a frequency outside the user's visually perceptive range, but with a visually perceptible cycle. The cycle is initiated by a strobe rate very slow and perceptible to the user and then ramp up rapidly to over 30 cycles per second for the dosage period. The cycle is terminated by ramping down the strobe rate from above 30 cycles per second to a slower, visually perceptible level at the end of the dosage period. The instant abstract is neither intended to define the invention disclosed in this specification nor intended to limit the scope of the invention in any way.

### RELATED APPLICATIONS

The present invention claims priority benefit from the following, previously filed applications:

U.S. Pat. No. 6,828,576, issued on Dec. 7, 2004, claiming benefit of Provisional Application No. 60/477,685, filed on Jun. 11, 2003;

U.S. Ser. No. 10/977,531 filed on Oct. 29, 2004 and claiming benefit of Provisional Application No. 60/477,685, filed on Jun. 11, 2003; and

U.S. Ser. No. 10/633,920, filed on Avg. 4, 2003 and claiming benefit of Provisional Application No. 60/477,685, filed on Jun. 11, 2003.

The entire disclosure and contents of the above applications are hereby incorporated by reference as if fully rewritten.

### BACKGROUND OF THE INVENTION

1. Field of the Invention

This invention relates to a therapeutic light systems and, more specifically, to an improvement method of operating such systems to allow for improved effectiveness without the known detrimental side effects.

2. Description of the Related Art

Light therapy or phototherapy consists of exposure to specific wavelengths of light using lasers, LEDs, fluorescent lamps, dichromic lamps or very bright, full-spectrum light, for a prescribed amount of time. It has proven effective in treating Acne vulgaris, seasonal affective disorder (SAD), and for some people it has ameliorated delayed sleep phase syndrome. It has recently been shown effective in non-seasonal depression. Proponents claim demonstrable benefits for skin conditions such as psoriasis and, more controversially, a degree of “skin rejuvenation.”

However, in order to elicit a therapeutic effect, a patient's exposure to any selected wavelength of light is accompanied by one main side-effect: increased thermal retention. The exposure of a patient's skin to the use of LEDs, fluorescent lamps, dichromic lamps or very bright, full-spectrum light over a period of time results in thermal:effects which themselves can be deleterious. Consequently, give the method of illumination, the selected wavelength range, the condition to be treated, and variables of the patient's skin, there is an exposure range called “effective irradiance” and a dose called “biologically effective dose.”

Consequently, any process designed to modify cell activity using light sources without thermal can benefit through the use of a photomodulation technique.

However, the use of modulated light on a human also has known deleterious effects, primarily known as ‘flicker vertigo’. Flicker vertigo can be unpleasant and produce dangerous reactions for the viewer. A light flickering at the rate of 4 to 20 cycles per second can produce such an effect that can result in nausea, vomiting, or, on rare occasions, unconsciousness. It is a condition well known to pilots, especially helicopter pilots more than those in fixed wing, propeller-driven aircraft, but both have reported such conditions. Flicker vertigo is also known to develop when viewing rotating beacons, strobe lights, or reflections of these off water or the clouds.

Usually, symptoms are mild and will stop when the source of the flickering goes away. Sometimes the individual is unaware of flicker vertigo. Flicker vertigo should not be confused with vertigo, which is a disorder of the inner ear. An individual suffering from vertigo has a sensation of spinning or believing the surroundings are spinning.

The Flight Safety Foundation (FSF) describes flicker vertigo as ““an imbalance in brain cell activity caused by exposure to low-frequency flickering (flashing) of a relative bright light.””. According to the FSF, the eye and the brain act together to perceive flickering light, and the activities of the retina (at the back of the eye where final images are formed) and the brain are synchronized as part of the visual process. If the flicker frequency is high enough, the system will perceive the light as steady. The critical speed of flickering will vary from person to person.

One way to eliminate the effects of flicker vertigo is to modulate the light or images above the frequency at which flicker becomes invisible. This flicker fusion rate is defined as the frequency at which an intermittent light stimulus appears to be completely steady to the observer. This flicker fusion rate is dependent on the level of illumination and proportional to the amount of modulation; if brightness is constant, a brief flicker will manifest a much lower threshold frequency than a long flicker. The threshold also varies with brightness (it is higher for a brighter light source) and with location on the retina where the perceived image falls: the rod cells of the human eye have a faster response time than the cone cells, so flicker can be sensed in peripheral vision at higher frequencies than in foveal vision. The flicker fusion threshold also is higher for a fatigued observer.

Consequently, in order to improve phototherapeutic techniques through the use of photomodulation, a need exists to prevent the deleterious effects thereof.

### SUMMARY OF THE INVENTION

It is therefore an object of the present invention to provide an improved therapeutic light system.

It is a feature of the present invention to provide an improved therapeutic light system that provides for the application of therapeutically specific radiation in a modulated fashion outside the eye perceptive range.

Briefly described according to one embodiment of the present invention, a non-thermal low dose light emitting diode (LED) array provides for the application of therapeutically specific radiation. The array delivers radiation to the intended target surface (anticipated as being a person's face, body or skin) at a controlled intensity. The intensity is controlled with a specific sequence of pulsing by pulse driving a matrix-arrayed LED One way to eliminate the effects of flicker vertigo is to modulate the light or images above the frequency at which flicker becomes invisible. This flicker fusion rate is defined as the frequency at which an intermittent light stimulus appears to be completely steady to the observer. This flicker fusion rate is dependent on the level of illumination and proportional to the amount of modulation; if brightness is constant, a brief flicker will manifest a much lower threshold frequency than a long flicker. The threshold also varies with brightness (it is higher for a brighter light source) and with location on the retina where the perceived image falls: the rod cells of the human eye have a faster response time than the cone cells, so flicker can be sensed in peripheral vision at higher frequencies than in foveal vision. The flicker fusion threshold also is higher for a fatigued observer.

An advantage of the present invention is that is that a biologically effective dose of an effective irrradiance of a therapeutic light source can be achieved.

Another advantage of the present invention is that phototherapy can be achieved without thermal effects on the patient.

Yet another advantage of the present invention is that modulated pulse can be achieved without any flicker vertigo effects on the user.

### BRIEF DESCRIPTION OF THE DRAWINGS

The advantages and features of the present invention will become better understood with reference to the following more detailed description and claims taken in conjunction with the accompanying drawings, in which like elements are identified with like symbols, and in which:

FIG. 1 is a perspective illustration of a non-thermal low dose light emitting diode (LED) array provides for the application of therapeutically specific radiation according to an exemplary embodiment of the present invention;

FIG. 2 is a electrical schematic of an LED multiplexing driver circuit for use therewith.

### DESCRIPTION OF THE PREFERRED EMBODIMENTS

The best mode for carrying out the invention is presented in terms of its preferred embodiment, herein depicted within the Figures

### 1. Detailed Description of the Figures

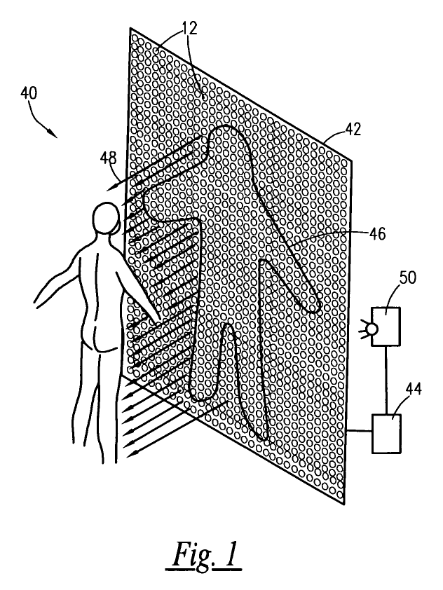

The present invention provides for an array of light emitting diodes (LED) as exemplified in FIG. 1. As is well-known in the art, LED's have generally small and round physical characteristics. Accordingly, a plurality of LED's can be arranged into matrixes of virtually any type of shape. A plurality of LED's 12 is arranged into a matrix 14. When energized by a power and control system 18 the matrix 14 projects a light illumination pattern 16. It is preferred that the matrix 14 shape conform to the intended target of the UV illumination pattern 16, a user P. In this fashion the present embodiment provides superior efficiencies compared to prior art fluorescent tube tanning systems, which necessarily consist of generally rectilinear arrays of long linear fluorescent tubes.

The array of LED's may be in communication with a sensor system 50 that detects shape parameters of the person P and determines the profile pattern 46 according to those parameters. For example, those LED's 12 located beyond the height and/or width of the person may not be illuminated. Thus, the replacement of UV fluorescent tubes with UV LED's allows for superior lighting efficiencies, since no energy or lighting resources must be expended to illuminate regions beyond the human profile shape 46.

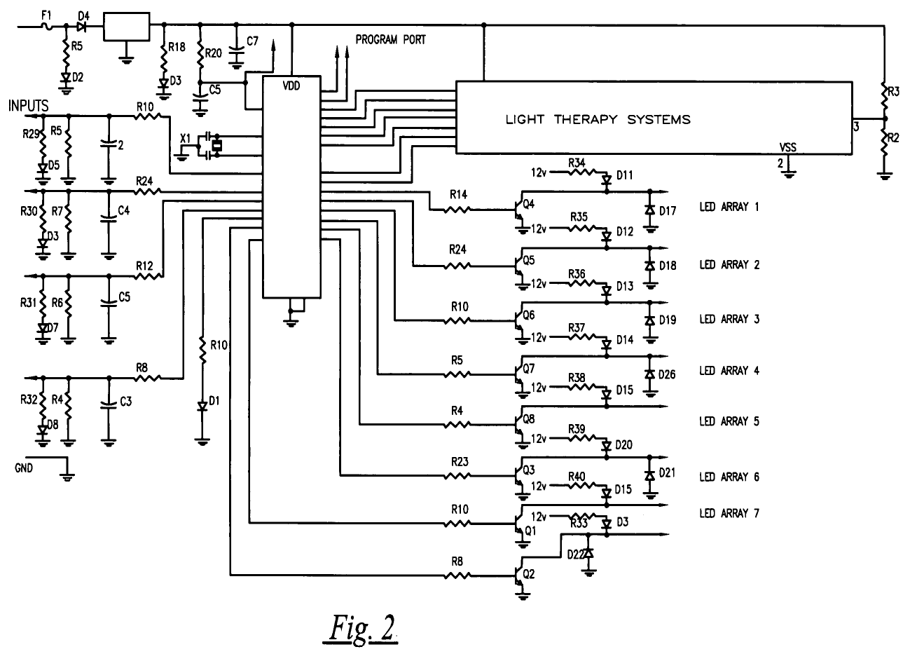

As best described in conjunction with FIG. 2, it is intended that the control system of the present invention would allow the LED array to provide an average radiation application can be controlled within an intended range. Further, a uniformed cycling of pulsing of the LED array is intended at greater than 30 Hz in a time multiplexing fashion. In such a manner, each LED is ON for a certain duration only, intended to be beyond a critical frequency does not appear to be flickering. This critical frequency is sometimes called the minimum refresh rate (television images actually refresh every 1/30 sec.). The recommended rate is 100 Hz to avoid flickering when the display is vibrating. For thermal considerations, 1 kHz is recommended.

Referring more specifically in conjunction with FIG. 2, a common-cathode line is connected to the current sinks while the common-anode line is driven by source drivers. The source drivers act like switches, allowing only one column/row of LEDs to be active at any one time. Source drivers can be as simple as a PNP transistor to digitally controlled drivers (TD6238x from Toshiba).

Additional, pulse width modulation (PWM) can be used to control LED brightness during the pulsing. By controlling the perceived brightness of an LED by turning it ON for only a certain period of time t. If the LED is on for time t over a period T, then the duty factor (DF) is t/T. A relationship between DF and perceived brightness or a relationship between average current and brightness where average current is the peak current multiplied by the DF.

### 2. Operation of the Preferred Embodiment

To use the present invention, the teachings of the present invention can be incorporated in conjunction with a number of different types of phototherapy techniques. However, such devices functioning in a multiplexed manner to provide an effective irradiance in a multiplexed manner at frequencies above those perceptible by foveal vision of the user can lead to problems. By not being able to perceive the functioning of the particular photottherapy, a user may easily overdose or underdose. Consequently, in operation it is intended that the particular phototherapy device or technique will have a perceivable cycle in which the multiplex/strobe of the LEDs begin at a strobe rate very slow and perceptible to the user and then ramp up rapidly to over 30 cycles per second for the dosage period. To terminate the session, a declining multiplex/strobe rate would also be visually perceptible to the user. Such a method would allow the user visually identifiable queues to identify the initiation and termination of the operational cycle.

The foregoing descriptions of specific embodiments of the present invention have been presented for purposes of illustration and description. They are not intended to be exhaustive or to limit the invention to the precise forms disclosed, and obviously many modifications and variations are possible in light of the above teaching. The embodiments were chosen and described in order to best explain the principles of the invention and its practical application, to thereby enable others skilled in the art to best utilize the invention and various embodiments with various modifications as are suited to the particular use contemplated. It is intended that the scope of the invention be defined by the Claims appended hereto and their equivalents. Therefore, the scope of the invention is to be limited only by the following claims.

Having thus described the invention what is claimed as new and desired to be secured by Letters Patent is as follows:

1. In a therapeutic light system that provides for the application of therapeutically specific radiation, wherein the improvement comprises modulating said therapeutically specific radiation at a frequency outside the user's visually perceptive range.

2. The improvement of claim 1, wherein said therapeutic light system comprises a non-thermal low dose light emitting diode (LED) array provides for the application of said therapeutically specific radiation.

3. The improvement of claim 2, wherein said array delivers radiation to the intended target surface at a controlled intensity, wherein the intensity is controlled with a specific sequence of pulsing by pulse driving a matrix-arrayed LED.

4. In the therapeutic light system of claim 3, wherein the improvement further comprises:
providing said therapeutically specific radiation in a visually perceivable cycle.

5. The improvement of claim 4, wherein said visually perceivable cycle is initiated by the strobing of said LED array at a strobe rate very slow and perceptible to the user and then ramp up rapidly to over 30 cycles per second for a dosage period.

6. The improvement of claim 5, wherein said visually perceivable cycle is terminated by a declining multiplex/strobe rate that is visually perceptible to the user.

7. A therapeutic light system comprising:
a matrix array of light emitting diodes (LED) for projecting a light illumination pattern;
a power and control system for modulating said projected light in a manner to allow said LED array to provide a controlled average radiation application within an intended range.

8. The therapeutic light system of claim 7, wherein said power and control system further comprise means for providing pulsing of said LED array at a rage greater than 30 Hz in a time multiplexing fashion.

9. The therapeutic light system of claim 8, wherein said power and control system further comprises means for providing a perceivable cycle in which the multiplex/strobe of the LEDs begin at a strobe rate very slow and perceptible to the user and then ramp up rapidly to over 30 cycles per second for the dosage period.

10. The therapeutic light system of claim 9, wherein said power and control system further comprises means for providing a perceivable termination of said cycle in which the rate of multiplex/strobe of the LEDs declining to below a rate that is visually perceptible to the user.
